(19) United States
(12) Patent Application Publication
(10) Pub. No.: US 2022/0233468 A1
(43) Pub. Date: Jul. 28, 2022

(54) METHODS FOR TREATING CORONAVIRUS INFECTION

(71) Applicant: Shauh-Der YEH, New Taipei City (TW)

(72) Inventor: Shauh-Der YEH, New Taipei City (TW)

(21) Appl. No.: 17/586,206

(22) Filed: Jan. 27, 2022

Related U.S. Application Data
(60) Provisional application No. 63/142,113, filed on Jan. 27, 2021.

(51) Int. Cl. A61K 31/12; A61P 31/14

(52) U.S. Cl. CPC A61K 31/12; A61P 31/14

(57) ABSTRACT

The present invention provides methods for treating or preventing coronavirus infection, or a coronavirus-associated disease, condition, or symptom in a subject. In particular, the methods comprise administering a transmembrane protease serine 2 (TMPRSS2) inhibitor, or a pharmaceutically acceptable salt or tautomer thereof.

### FIELD OF THE INVENTION

The present invention relates to transmembrane protease serine 2 (TMPRSS2) inhibitors for use in treating or preventing coronavirus infection. The present invention in particular relates to the use of TMPRSS2 inhibitors for treating or preventing coronavirus infection, or a coronavirus-associated disease, condition or symptom in a subject.

### BACKGROUND OF THE INVENTION

Coronaviruses (CoVs) belong to the Coronaviridae family and the Orthocoronaviridae subfamily, and are enveloped, positive-sense, single-stranded RNA viruses that primarily infect respiratory and gastrointestinal tracts of a wide range of animal species. A CoV particle comprises at least the four canonical structural proteins: the E (envelope) protein, M (membrane) protein, N (nucleocapsid) protein, and S (spike) protein, wherein the S protein mediates the binding of the virus to host cellular receptors and subsequent fusion between the viral and host cell membranes (R. J. G. Hulswit, el al., Advances in Virus Research 2016, 96:29-57; Dewald Schoeman and Burtram C. Fielding, Virology Journal 2019, 16(1):69). CoVs were known to cause mild illnesses in humans, such as common colds, and were considered as nonfatal viruses until they caused three major outbreaks of severe respiratory disease in recent years, i.e., severe acute respiratory syndrome (SARS) in 2002 to 2003, Middle East respiratory syndrome (MERS) in 2012, and coronavirus disease 2019 (COVID-19).

COVID-19 is caused by a new coronavirus, SARS-CoV-2, which was first identified in Wuhan, China, in December 2019, was found to be closely related to SARS-CoV, and may lead to severe illness or even death. The symptoms of COVID-19 may appear 2 to 14 days after exposure to the virus, and the clinical spectrum thereof varies from asymptomatic or paucisymptomatic forms, mild symptoms (such as fever, dry cough, myalgia, headache and diarrhea) to severe conditions (such as dyspnea, hypoxia, pneumonia and systemic complications) (Marco Cascella, et al., Features, Evaluation and Treatment Coronavirus (COVID-19), StatPearls: Treasure Island, 2020).

According to the sequence alignment and homology modeling results, SARS-CoV and SARS-CoV-2 share a highly conserved receptor-binding domain, a domain of S protein, and approximately 76% amino acid similarity in their S proteins (Dong N, et al., F1000Research 2020, 9:121). For SARS-CoV, host cell entry depends on the binding of the S1 subunit of the S protein to a host cellular receptor, angiotensin-converting enzyme 2 (ACE2), and the S protein priming by cellular proteases, endosomal cysteine proteases cathepsin B and L (CatB/L) and transmembrane protease serine 2 (TMPRSS2), which involves S protein cleavage and induces virus-cell membrane fusion at the host cell surface. It was demonstrated that SARS-CoV-2, like SARS-CoV, employs ACE2 as the cellular entry receptor and CatB/L and TMPRSS2 for S protein priming, and showed that an anti-ACE2 antibody or antibody responses raised against SARS-CoV during infection or vaccination can inhibit cell entries of SARS-CoV and SARS-CoV-2 in vitro; camostat mesylate (a TMPRSS2 inhibitor) partially blocks cell entries of SARS-CoV and SARS-CoV-2; and the combination of camostat mesylate and E-64d (a CatB/L inhibitor) can fully block the cell entries of SARS-CoV and SARS-CoV-2 (Hoffmann M., et al., Cell 2020, 181:271-280; Xiuyuan Ou, et al., Nature Communications 2020, 11(1):1620).

TMPRSS2 is a cell surface serine protease and is well known for its predominant expression in prostate epithelium cells and overexpression in metastatic prostate cancers, while its normal physiological role(s) is unknown (1 M. Lucas, et al., Cancer Discovery 2014, 4(11):1310-1325). CoV S protein priming by TMPRSS2 is shown to be crucial for viral entry, spread and pathogenesis in the infected host (Hoffmann M., et al., Cell 2020, 181:271-280; S. Matsuyama, et al., Journal of Virology 2010, 84:12658-12664; N. Iwata-Yoshikawa, et al., Journal of Virology 2019, 93: e01815-18), which suggests that TMPRSS2 might be a potential therapeutic target for SARS-CoV-2 infection. Camostat, Nafamostat and Aprotinin are serine protease inhibitors clinically approved for, e.g., the treatment of pancreatitis and prophylactic use to reduce perioperative blood loss, and are shown to block the SARS-CoV-2 entry and reduce the infection rate in vitro, which might constitute a treatment option for COVID-19 (Hoffmann M., et al., Cell 2020, 181:271-280; M. Yamamoto, et al., Viruses 2020, 12(6): 629; D. Bestle, et al., Life Science Alliance 2020, 3(9): e202000786).

Despite several pharmaceutical agents and vaccines potentially useful in the treatment and/or prophylaxis of COVID-19 being continuously researched and developed, there remains an urgent need to develop new therapeutic and prophylactic therapies for COVID-19 and coronavirus-associated diseases.

### SUMMARY OF THE INVENTION

The present disclosure relates to compounds that are capable of inhibiting the expression of TMPRSS2 and may be used in the treatment and prophylaxis of coronavirus infection. The present disclosure relates to the use of TMPRSS2 inhibitors for treating or preventing coronavirus infection, or a coronavirus-associated disease, condition or symptom in a subject.

The disclosure provides a method for treating or preventing coronavirus infection in a subject, comprising administering a therapeutically effective amount of a TMPRSS2 inhibitor to the subject. The disclosure also provides a method for inhibiting, blocking or preventing coronavirus entry into cells of a subject, comprising administering a therapeutically effective amount of a TMPRSS2 inhibitor to the subject. The disclosure further provides a method for reducing the coronavirus infection rate in a subject, comprising administering a therapeutically effective amount of a TMPRSS2 inhibitor to the subject. The disclosure further provides a method for inhibiting, blocking or reducing coronavirus transmission, comprising administering a therapeutically effective amount of a TMPRSS2 inhibitor to a subject. The disclosure further provides a method for preventing, treating or ameliorating a coronavirus-associated disease, condition or symptom in a subject, comprising administering a therapeutically effective amount of a TMPRSS2 inhibitor to the subject.

In some embodiments, the TMPRSS2 inhibitor is a compound of formula I:

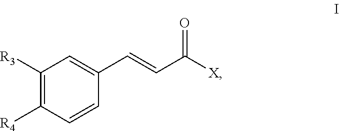

or pharmaceutically acceptable salts thereof, wherein X, R₃ and R₄ are as defined in U.S. Pat. No. 8,236,852 B2 (Shih, et al.) and U.S. Pat. No. 8,710,272 B2 (Shih, el al.), incorporated herein by reference.

In some embodiments, the TMPRSS2 inhibitor is a compound of formula IIa, IIb, or VI:

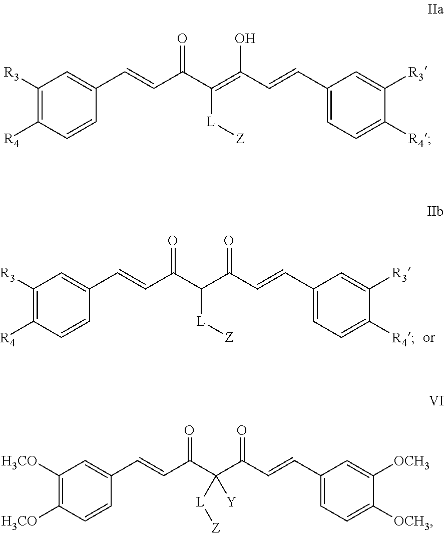

or pharmaceutically acceptable salts thereof, wherein R₃, R₄, R₃′, R₄′, L, Z and Y are as defined in U.S. Pat. No. 8,236,852 B2 (Shih, et al.) and U.S. Pat. No. 8,710,272 B2 (Shih, et al.), incorporated herein by reference.

In certain embodiments, the TMPRSS2 inhibitor is Compound A or Compound B, or pharmaceutically acceptable salts or tautomers thereof.

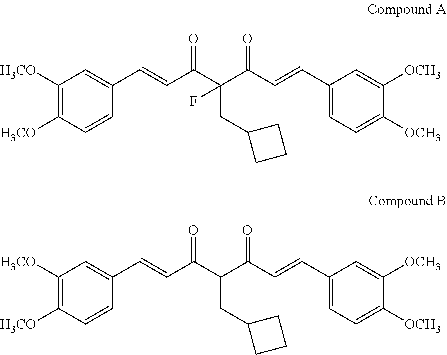 [SMILES: COc1ccc(/C=C/C(=O)C(F)(CC2CCC2)C(=O)/C=C/c2ccc(OC)c(OC)c2)cc1OC ; COc1ccc(/C=C/C(=O)C(CC2CCC2)C(=O)/C=C/c2ccc(OC)c(OC)c2)cc1OC]

In some embodiments, the method further comprises administering a therapeutically effective amount of one or more additional antiviral agents to the subject. In some embodiments, the coronavirus is SARS-CoV, SARS-CoV-2 or MERS-CoV. In some embodiments, the coronavirus-associated disease is SARS, COVID-19 or MERS.

### BRIEF DESCRIPTION OF THE INVENTION

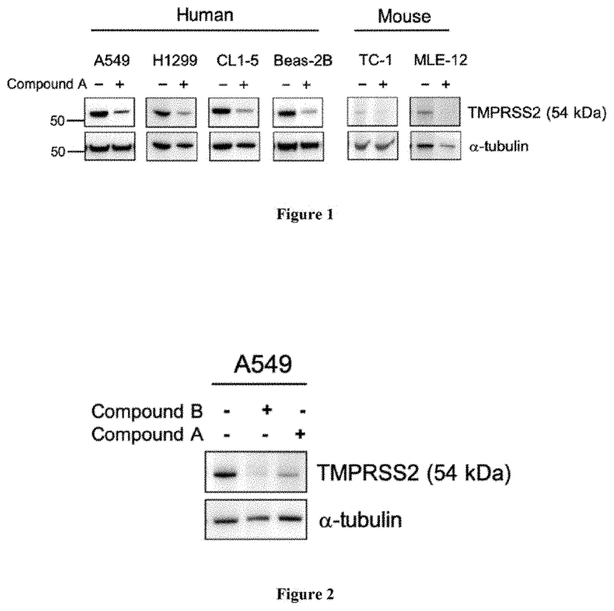

FIG. 1 shows the effect of Compound A on TMPRSS2 expression in different cell lines.

FIG. 2 shows the effects of Compound A and Compound B on TMPRSS2 expression in A549 cell lines.

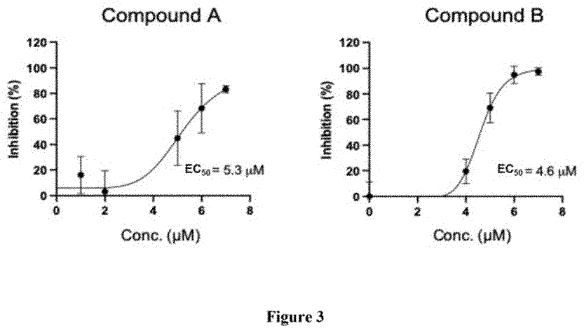

FIG. 3 shows the effects of Compound A and Compound B on cellular entry of SARS-CoV-2.

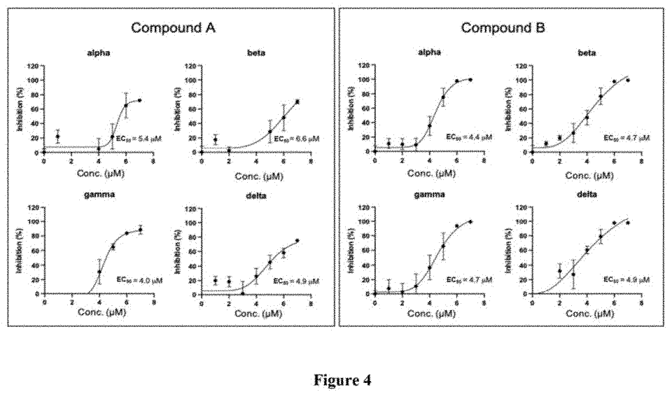

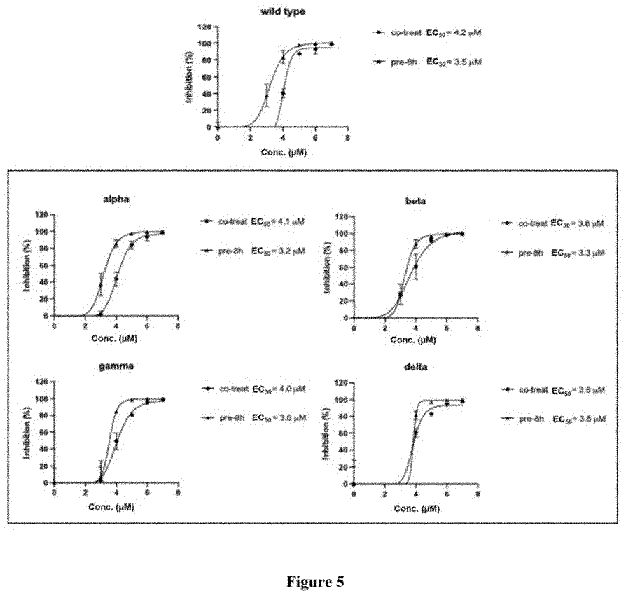

FIG. 4 shows the effects of Compound A and Compound B on cellular entry of SARS-CoV-2 variants.

FIG. 5 shows the prophylactic effect of Compound B on cellular entry of SARS-CoV-2.

### DETAILED DESCRIPTION OF THE INVENTION

Unless otherwise defined, all technical and scientific terms used in connection with the present invention have the meanings that are commonly understood by persons of ordinary skill in the art. In addition, the following definitions are set forth to illustrate and define the meaning and scope of the various terms used to describe the invention, and the following description sets forth exemplary methods, parameters and the like. It should be recognized, however, that such description is not intended as a limitation to the scope of the present invention, but is instead provided as a description of exemplary embodiments.

The terms “a” and “an” and “the” as used herein may mean one or more than one, unless otherwise indicated herein or clearly contradicted by context.

The term “about” or “around” as used herein refers to a value per se or ±20% of the recited value. In some embodiments, the term refers to values oft 15%, ±10%, ±5%, ±2% and ±1% of the recited value.

The term “coronavirus” or “CoV” as used herein refers to any member of Coronaviridae family, including, but not limited to, the wild-type coronavirus, naturally-occurring coronavirus variants and coronavirus variants generated in a laboratory. In some embodiments, the term “coronavirus” or “CoV” refers to SARS-CoV or SARS-CoV-2. In some embodiments, the term “SARS-CoV-2” refers to wild-type SARS-CoV-2 or SARS-CoV-2 variants, including, but not limited to, Alpha variant (BA 0.1.7), Beta variant (B.1.351), Gamma variant (P.1), Delta variant (B.1.617.2), and Omicron variant (B.1.1.529).

The term “subject” as used herein refers to animals, preferably mammals, including, but not limited to, rodents, bats, carnivores, ungulates and primates. In some embodiments, the term “subject” refers to “humans.”

The term “treating,” “treatment” or “therapeutic” as used herein refers to partially or completely delaying, controlling, stabilizing, interrupting, inhibiting, ameliorating, alleviating, relieving, reducing, decreasing, eliminating, arresting or stopping the transmission, progression and/or severity of infections, diseases, conditions and/or symptoms associated with coronaviruses.

The term “preventing,” “prevention,” “prophylaxis” or “prophylactic” as used herein refer to partially or completely precluding, averting, obviating, interrupting, inhibiting, reducing, arresting and/or stopping the occurrence or development of infections, diseases, conditions and/or symptoms associated with coronaviruses.

The term “presymptomatic” as used herein refers to a subject that is infected with coronaviruses and has not yet showed or developed symptoms.

The term “asymptomatic” as used herein refers to a subject that is infected with coronaviruses, but does not show or develop symptoms.

The term “paucisymptomatic” as used herein refers to a subject that is infected with coronaviruses and only shows or develops one symptom, or shows or develops one or more mild symptoms that may not be noticed or reported.

The term “therapeutically effective amount” as used herein refers to an amount or dose of the compounds or agents of the invention, which upon single or multiple doses administration to a subject, provides the desired pharmacological, therapeutic or prophylactic effect in the subject, or sufficient to decrease one or more conditions and/or symptoms associated with coronaviruses. A therapeutically effective amount also can include a range of amounts and may vary depending on the pharmacokinetic and pharmacodynamic parameters of compounds administered, the state, progression or severity of the disease treated, the age, weight and relative health of the subject, the route and form of administration, the response of the subject and/or the dose regimen selected.

The term “pharmaceutically acceptable salt” as used herein refers to a salt of a compound that is pharmaceutically acceptable and that possesses the desired pharmacological activity and effectiveness of the parent compound or can be converted to a biological active form in the body of the subject. Pharmaceutically acceptable salts include both acid and base addition salts. Examples of pharmaceutically acceptable salts include, but are not limited to, hydrochlorate, phosphate, diphosphate, hydrobromate, iodide, sulfate, methanesulfonate, nitrate, malate, maleate, fumarate, tartrate, succinate, citrate, acetate, trifluoroacetate, lactate, mesylate, benzoate, salicylate, stearate, and alkanoate; and salts formed when an acidic proton present in the parent compound is replaced by a cation including, but not limited to, sodium ion, potassium ion, magnesium ion, calcium ion, and ammonium cation.

The present invention relates to the use of TMPRSS2 inhibitors for treating or preventing coronavirus infection, or a coronavirus-associated disease, condition or symptom in a subject.

One aspect of the present invention is a method for treating or preventing coronavirus infection in a subject, comprising administering a therapeutically effective amount of a TMPRSS2 inhibitor to the subject. One aspect of the present invention is a method for preventing, treating or ameliorating a coronavirus-associated disease, condition or symptom in a subject, comprising administering a therapeutically effective amount of a TMPRSS2 inhibitor to the subject. One aspect of the present invention is a method for inhibiting, blocking or preventing coronavirus entry into cells of a subject, comprising administering a therapeutically effective amount of a TMPRSS2 inhibitor to the subject. One aspect of the present invention is a method for reducing the coronavirus infection rate in a subject, comprising administering a therapeutically effective amount of a TMPRSS2 inhibitor to the subject. One aspect of the present invention is a method for inhibiting, blocking or reducing coronavirus transmission, comprising administering a therapeutically effective amount of a TMPRSS2 inhibitor to the subject. One aspect of the present invention is a method for reducing the risk of being infected with coronaviruses or developing a coronavirus-associated disease, condition or symptom in a subject, comprising administering a therapeutically effective amount of a TMPRSS2 inhibitor to the subject.

A further aspect of the invention is the use of a TMPRSS2 inhibitor in the manufacture of a medicament for treating or preventing coronavirus infection in a subject. A further aspect of the invention is the use of a TMPRSS2 inhibitor in the manufacture of a medicament for preventing, treating or ameliorating a coronavirus-associated disease, condition or symptom in a subject. A further aspect of the invention is the use of a TMPRSS2 inhibitor in the manufacture of a medicament for inhibiting, blocking or preventing coronavirus entry into cells of a subject. A further aspect of the invention is the use of a TMPRSS2 inhibitor in the manufacture of a medicament for reducing the coronavirus infection rate in a subject. A further aspect of the invention is the use of a TMPRSS2 inhibitor in the manufacture of a medicament for inhibiting, blocking or reducing coronavirus transmission in a subject. A further aspect of the invention is the use of a TMPRSS2 inhibitor in the manufacture of a medicament for reducing the risk of being infected with coronaviruses or developing a coronavirus-associated disease, condition or symptom in a subject.

In some embodiments, the subject has a coronavirus infection or a coronavirus-associated disease, condition or symptom. In some embodiments, the subject has a presymptomatic, asymptomatic or paucisymptomatic coronavirus infection. In some embodiments, the subject is at risk of being infected with coronaviruses or developing a coronavirus-associated disease, condition or symptom.

In some embodiments, the coronavirus is MERS-CoV, SARS-CoV or SARS-CoV-2. In some embodiments, the coronavirus-associated disease, condition or symptom includes MERS, SARS, COVID-19, fever, dry cough, sore throat, headache, muscle pain, fatigue, gastrointestinal symptoms (e.g., abdominal pain, diarrhea and vomiting), neurological symptoms (e.g., new loss of taste or smell, confusion and seizure), tachypnea (>30 breaths/min), hypoxia (Sp02<90% on room air), dyspnea, respiratory failure, pneumonia, acute respiratory distress syndrome (ARDS), systemic complications, and multiorgan failure. In some embodiments, the coronavirus-associated disease is MERS, SARS or COVID-19.

In some embodiments, the TMPRSS2 inhibitor is a compound of formula I:

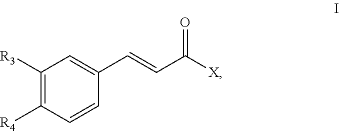

or pharmaceutically acceptable salts thereof, wherein X, R₃ and R₄ are as defined in U.S. Pat. No. 8,236,852 B2 (Shih, et al.) and U.S. Pat. No. 8,710,272 B2 (Shih, et al.), incorporated herein by reference.

In some embodiments, the TMPRSS2 inhibitor is a compound of formula IIa, IIb, or VI:

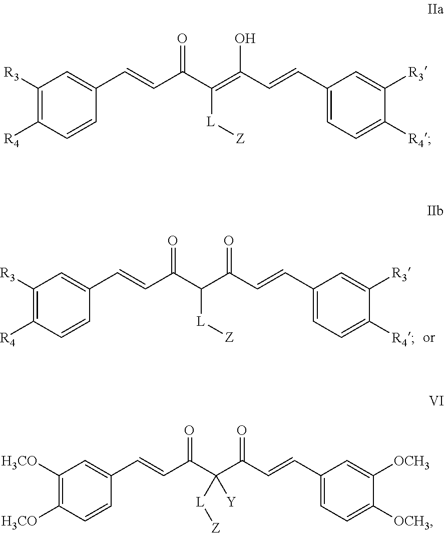

or pharmaceutically acceptable salts thereof, wherein R₃, R₄, R₃′, R₄′, L, Z and Y are as defined in U.S. Pat. No. 8,236,852 B2 (Shih, et al.) and U.S. Pat. No. 8,710,272 B2 (Shih, et al.), incorporated herein by reference.

In certain embodiments, the TMPRSS2 inhibitor is Compound A, or a pharmaceutically acceptable salt thereof. Compound A is 4-fluoro-4-cyclobutylmethyl-1,7-bis(3,4-dimethoxyphenyl)hepta-1,6-diene-3,5-dione and has the structure:

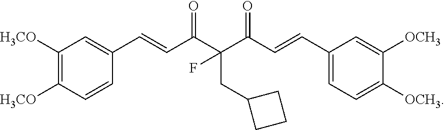 [SMILES: COc1ccc(/C=C/C(=O)C(F)(CC2CCC2)C(=O)/C=C/c2ccc(OC)c(OC)c2)cc1OC]

In certain embodiments, the TMPRSS2 inhibitor is Compound B, or a pharmaceutically acceptable salt or tautomer thereof. Compound B is 4-(cyclobutylmethyl)-1,7-bis(3,4-dimethoxyphenyl)hepta-1,6-diene-3,5-dione and has the structure:

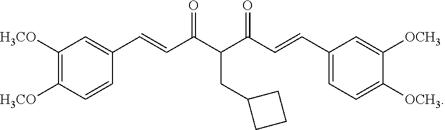 [SMILES: COc1ccc(/C=C/C(=O)C(CC2CCC2)C(=O)/C=C/c2ccc(OC)c(OC)c2)cc1OC]

Compound B is a curcumin derivative and was granted for the treatment of X-linked spinal and bulbar muscular atrophy (Kennedy's disease) in Europe (Public summary of opinion on orphan designation, EMA/COMP/254125/2016). Compound A is derived from Compound B and has improved chemical and metabolic stability.

In addition, Compound B may exhibit the phenomenon of tautomerism. For example, Compound B may exist as an equilibrium between enol-ketone and diketone forms (as shown below). Tautomeric isomers are in equilibrium with one another. Regardless of which tautomer is shown, and regardless of the nature of the equilibrium among tautomers, Compound B is understood by persons of ordinary skill in the art to comprise both.

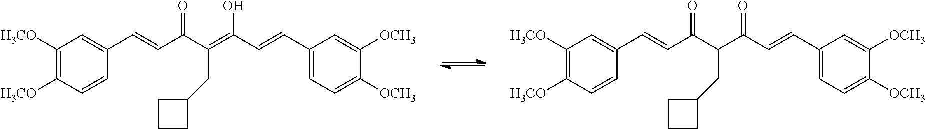 [SMILES: COc1ccc(/C=C/C(=O)/C(CC2CCC2)=C(O)/C=C/c2ccc(OC)c(OC)c2)cc1OC ; COc1ccc(/C=C/C(=O)C(CC2CCC2)C(=O)/C=C/c2ccc(OC)c(OC)c2)cc1OC]

The synthesis and characterization of Compound A and Compound B and their uses in treating medical conditions, including androgen associated conditions, Kennedy's disease and prostate cancer, have been described in U.S. Pat. No. 8,236,852 B2 (Shih, et al.) and U.S. Pat. No. 8,710,272 B2 (Shih, et al.), which is incorporated herein by reference in its entirety for all purposes.

Another aspect of the present invention is an in vitro or in vivo method for modulating TMPRSS2 expression in a target cell, comprising contacting an effective amount of Compound A or Compound B, or a pharmaceutically acceptable salt or tautomer thereof, to the target cell. In some embodiments, the target cell is a mammalian cell. In some embodiments, the target cell is a human cell. In some embodiments, the target cell includes lung cells, corneal cells, esophagus cells, colon cells, pancreatic cells, gallbladder cells, kidney cells and prostate cells. In some embodiments, the target cell includes nasal epithelial cells, nasal ciliated cells, lung ciliated cells, lung epithelial cells, bronchial epithelial cells, alveolar epithelial cells and conjunctival cells.

The compounds of the invention may be formulated as a pharmaceutical composition/formulation or a medicament together with one or more pharmaceutically acceptable vehicles and/or additional medical agents. Examples of the pharmaceutically acceptable vehicles include, but are not limited to, carriers, diluents, disintegrants, surfactants, binders, pH modifiers, lubricants, bulking agents, glidant, adjuvants, excipients, solvents and buffer solutions.

In some embodiments, the compounds of the invention may be administered by any suitable routes, including topical, oral, nasal, intestinal, rectal, parenteral, transmucosal, transdermal, intradermal, subcutaneous, intramuscular, intramedullary, intrathecal, intravenous, intra-arterial, intraperitoneal and ophthalmic administration. In some embodiments, the compounds of the invention may be administered in any suitable dosage forms, including tablets, capsules, syrups, sprays, aerosols, inhalants, powders, gels, emulsions, suppositories, liposomes, microspheres, suspensions and injections.

In some embodiments, the compounds of the invention may be administered in combination with one or more active agents simultaneously, separately, or sequentially in any order. In some embodiments, the compounds of the invention and one or more active agents may be administered as part of the same pharmaceutical composition/formulation, or in separate pharmaceutical compositions/formulations. In some embodiments, the compounds of the invention may be administered prior to, at the same time as, subsequent to, or during the interval of the administration of one or more active agents. In some embodiments, the compounds of the invention may be administered in a single dose or a series of doses and/or at different intervals prior to, at the same time as, subsequent to, or during the interval of the administration of one or more active agents.

In some embodiments, the active agents may be any agents that are advantageously combined with the compounds of the invention. In some embodiments, the active agents can improve the therapeutic or prophylactic efficacy of the compounds of the invention and/or can counteract or reduce potential side effects associated with the compounds of the invention. Examples of the active agents include, but are not limited to, antiviral agents; an anti-MERS-CoV antibody, an anti-SARS-CoV antibody, an anti-SARS-CoV-2 antibody, or antigen binding fragments thereof; RNA synthesis inhibitors (such as RNA-dependent RNA polymerase (RdRp) inhibitors); protease inhibitors (such main protease (Mpro), 3C-like cysteine protease (3CLpro) and papain-like protease (PLpro)); virus-host cell binding/fusion inhibitors (such as S protein, ACE2, angioensin II receptor (AT2), CatB/L and TMPRSS2 inhibitors); viral replication inhibitors (such as 3Clpro, non-structural protein 12 (Nsp12) and Nsp15 inhibitors and small interfering RNA (siRNA)); siRNAs targeting the M, N and/or E genes of coronaviruses; an anti-inflammatory agent (such as corticosteroids and nonsteroidal anti-inflammatory (NSAID) agents); immunomodulatory agents (such as JAK inhibitors and IL-6/IL-6R antagonists); immune checkpoint modulators (such as anti-PD-1 and anti-PD-L1 antibodies); anti-infective agents; cytokines (such as interferons); an anti-diarrheal agent; and antioxidants.

It will be readily apparent to one of ordinary skill in the relevant arts that other suitable modifications and adaptations to the methods and applications described herein can be made without departing from the scope of any of the embodiments. The following examples are included herewith for purposes of illustration only and are not intended to be limiting.

### EXAMPLES

### Example 1: Effects of Compound A and Compound B on TMPRSS2

The effects of Compound A and Compound B on TMPRSS2 were investigated in A549 (human alveolar epithelial cell), H1299 (human non-small cell lung carcinoma cell), CL1-5 (human lung adenocarcinoma cell), Beas-2B (human normal bronchial epithelial cell), TC-1 (mouse lung epithelial cell) and MLE-12 (mouse lung epithelial cell) cell lines. The expression of TMPRSS2 in cells was analyzed by Western blotting, and a-tubulin was used as an internal control. As shown in FIGS. 1 and 2, Compound A and Compound B can inhibit the protein expression of TMPRSS2 in different cell lines after 18 to 24 hrs treatment, and Compound B exhibited greater TMPRSS2 inhibitory activity as compared to Compound A. These results suggest that Compound A and Compound B are capable of inhibiting the protein expression of TMPRSS2, and may block the CoV fusion or entry into host cells.

### Example 2: Effects of Compound A and Compound B on Viral Entry

The effects of Compound A and Compound B on viral entry was analyzed using a lentiviral pseudotype system (Hoffmann M., et al., Cell 2020, 181:271-280; Xiuyuan Ou, et al., Nature Communications 2020, 11(1):1620). Lentiviral pseudovirions that express SARS-CoV-2 S protein were generated by co-transfection human embryonic kidney (HEK) 293T cells with a lentiviral packaging plasmid, a lentiviral receptor plasmid that expresses green fluorescent protein (GFP) and luciferase reporter protein, and a plasmid that encodes SARS-CoV-2 S protein. The transfected HEK 293T cells produce pseudotyped lentiviral particles harboring SARS-CoV-2 S protein (“SARS-CoV-2 S pseudovirions”). SARS-CoV-2 S pseudovirions can infect cells that express ACE2 receptors. HEK 293T cells stably expressing ACE2 (HEK293T-ACE2) were inoculated with the SARS-CoV-2 S pseudovirions and treated with either Compound A or Compound B for 18 to 24 hrs. HEK293T-ACE2 cells untreated with Compound A or Compound B were also included as a control. After 18 to 24 hrs post-infection, the SARS-CoV-2 S pseudovirions were removed and the HEK293T-ACE2 cells were washed with PBS before fresh medium was added. After 48 hrs post-incubation, the HEK293T-ACE2 cells were lysed and the luciferase activity in cell lysates was measured. The luciferase activity was used to reflect the infection or cellular entry of SARS-CoV-2 S pseudovirions. EC₅₀ values were calculated as the effective concentration of a compound that caused a 50% inhibition of viral entry based on the resultant luciferase activity.

As shown in FIG. 3, both Compound A and Compound B showed inhibition of cellular entry of SARS-CoV-2 S pseudovirions, where Compound A exhibited an EC₅₀ value of around 5.3 μM and Compound B exhibited an EC₅₀ value of around 4.6 μM. Compound B exhibited a better inhibitory activity as compared to Compound A.

The effects of Compound A and Compound B on the cellular entry of SARS-CoV-2 variants were also assessed using pseudotyped lentiviral particles harboring S protein from SARS-CoV-2 Alpha variant (B.1.1.7), Beta variant (B.1.351), Gamma variant (P.1), and Delta variant (B.1.617.2), respectively. As shown in FIG. 4, both Compound A and Compound B showed inhibition of cellular entry of pseudovirions with Spike variants, where Compound A exhibited a better inhibitory activity against Gamma variant (P.1) (with an EC₅₀ value of around 4.0 μM) and Compound B exhibited comparable inhibitory activity against all variants (with EC₅₀ values of around 4.4 μM for Alpha variant, around 4.7 μM for Beta and Gamma variants, and around 4.9 μM for Delta variant).

The prophylactic effect of Compound B on the cellular entry of wild-type SARS-CoV-2 and variants was further assessed. HEK293T-ACE2 cells were first treated with Compound B. After 8 hrs incubation, the HEK293T-ACE2 cells were inoculated with pseudovirions harboring S protein from wild-typeSARS-CoV-2, Alpha variant (B.1.1.7), Beta variant (B.1.351), Gamma variant (P.1), and Delta variant (B.1.617.2), respectively. As shown in FIG. 5, pre-treatment of Compound B even showed better inhibition of cellular entry of pseudovirions either with wild-type Spike (with an EC₅₀ value of around 3.5 μM) or Spike variants (with EC₅₀ values of around 3.2 μM for Alpha variant, around 3.3 μM for Beta variant, around 3.6 μM for Gamma variant, and around 3.8 μM for Delta variant).

The data demonstrates that Compound A and Compound B are capable of inhibiting the SARS-CoV-2 fusion or entry into host cells, which suggest that they have antiviral capability against SARS-CoV-2 and may be used in the treatment and prophylaxis of SARS-CoV-2 infection or transmission.

It is to be understood that while various embodiments have been illustrated and described herein, the claims are not to be limited to the specific forms or arrangement of parts described and shown. In the specification, there have been disclosed illustrative embodiments and, although specific terms are employed, they are used in a generic and descriptive sense only, and not for purposes of limitation. Modifications and variations of the embodiments are possible in light of the above teachings. It is therefore to be understood that the embodiments may be practiced otherwise than as specifically described. All patents and publications discussed herein are incorporated by reference in their entirety.

1. A method for treating or preventing coronavirus infection in a subject, comprising administering a therapeutically effective amount of a TMPRSS2 inhibitor to the subject, wherein the TMPRSS2 inhibitor is a compound of formula I: 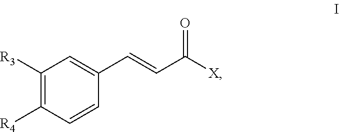
or a pharmaceutically acceptable salt thereof, wherein X, R₃ and R₄ are as defined in U.S. Pat. No. 8,236,852 B2 and U.S. Pat. No. 8,710,272 B2, incorporated herein by reference.

2. A method for inhibiting, blocking or preventing coronavirus entry into cells of a subject, comprising administering a therapeutically effective amount of a TMPRSS2 inhibitor to the subject, wherein the TMPRSS2 inhibitor is a compound of formula I: 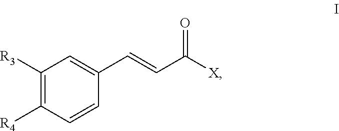
or a pharmaceutically acceptable salt thereof, wherein X, R₃ and R₄ are as defined in U.S. Pat. No. 8,236,852 B2 and U.S. Pat. No. 8,710,272 B2, incorporated herein by reference.

3. A method for inhibiting, blocking or reducing coronavirus transmission, comprising administering a therapeutically effective amount of a TMPRSS2 inhibitor to the subject, wherein the TMPRSS2 inhibitor is a compound of formula I: 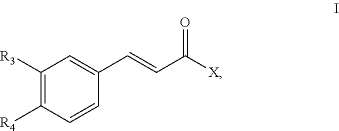
or a pharmaceutically acceptable salt thereof, wherein X, R₃ and R₄ are as defined in U.S. Pat. No. 8,236,852 B2 and U.S. Pat. No. 8,710,272 B2, incorporated herein by reference.

4. A method for reducing the risk of being infected with coronaviruses or developing a coronavirus-associated disease, condition or symptom in a subject, comprising administering a therapeutically effective amount of a TMPRSS2 inhibitor to the subject, wherein the TMPRSS2 inhibitor is a compound of formula I: 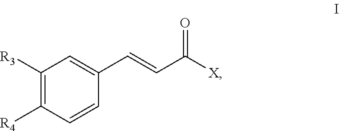
or a pharmaceutically acceptable salt thereof, wherein X, R₃ and R₄ are as defined in U.S. Pat. No. 8,236,852 B2 and U.S. Pat. No. 8,710,272 B2, incorporated herein by reference.

5. A method for preventing, treating or ameliorating a coronavirus-associated disease, condition or symptom in a subject, comprising administering a therapeutically effective amount of a TMPRSS2 inhibitor to the subject, wherein the TMPRSS2 inhibitor is a compound of formula I: 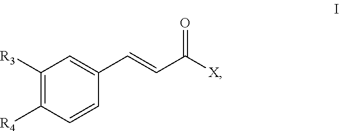
or a pharmaceutically acceptable salt thereof, wherein X, R₃ and R₄ are as defined in U.S. Pat. No. 8,236,852 B2 and U.S. Pat. No. 8,710,272 B2, incorporated herein by reference.

6. An in vitro or in vivo method for modulating TMPRSS2 expression in a target cell, comprising contacting an effective amount of a compound of formula I: 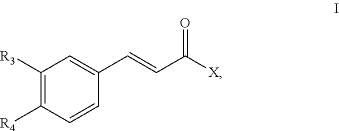
or a pharmaceutically acceptable salt thereof, wherein X, R₃ and R₄ are as defined in U.S. Pat. No. 8,236,852 B2 and U.S. Pat. No. 8,710,272 B2, incorporated herein by reference, wherein the target cell is selected from the group consisting of lung cells, corneal cells, esophagus cells, colon cells, pancreatic cells, gallbladder cells, kidney cells and prostate cells.

7. The method of claim 1, wherein the compound of formula I is a compound of formula IIa, IIb, or VI: 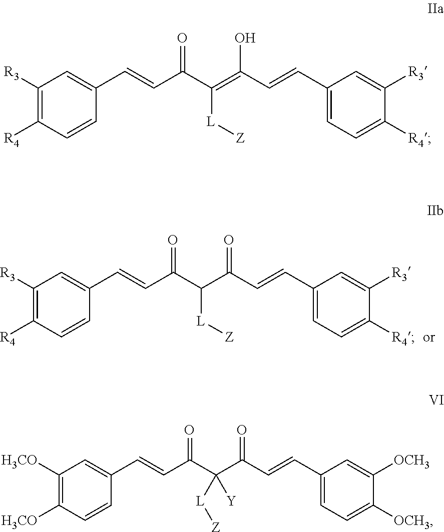
or a pharmaceutically acceptable salt thereof, wherein R₃, R₄, R₃′, R₄′, L, Z and Y are as defined in U.S. Pat. No. 8,236,852 B2 and U.S. Pat. No. 8,710,272 B2, incorporated herein by reference.

8. The method of claim 1, wherein the compound of formula I is Compound A having the structure: 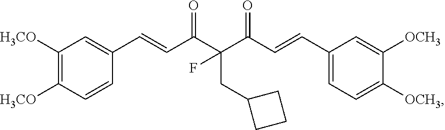 [SMILES: COc1ccc(/C=C/C(=O)C(F)(CC2CCC2)C(=O)/C=C/c2ccc(OC)c(OC)c2)cc1OC]
or a pharmaceutically acceptable salt thereof; or Compound B having the structure: 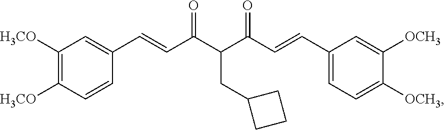 [SMILES: COc1ccc(/C=C/C(=O)C(CC2CCC2)C(=O)/C=C/c2ccc(OC)c(OC)c2)cc1OC]
or a pharmaceutically acceptable salt or tautomer thereof.

9. The method of claim 1, wherein the compound of formula I is administered topically, orally, nasally, intestinally, rectal, parenterally, transmucosally, transdermally, intradermally, subcutaneously, intramuscularly, intramedullaryly, intrathecally, intravenously, intraarterially, intraperitoneally, or ophthalmically.

10. The method of claim 1, wherein the compound of formula I is administered in the form of a tablet, capsule, syrup, spray, aerosol, inhalant, powder, gel, emulsion, suppository, liposome, microsphere, suspension, or injection.
